SEQUENCE LISTING (10 sequences)

SEQ ID NO: 1 - DNA, 19 bp, Artificial Sequence
  Forward primer
gcggtcccactcttcacag

SEQ ID NO: 2 - DNA, 21 bp, Artificial Sequence
  Reverse primer
gcccagaggctagtgttacca

SEQ ID NO: 3 - protein, 297 aa, Ljungan virus 87-012
  VP1
GLHSWGSEMDLVDSLDNPDEIQDNEEIQTQNVEAAQGEEAATEVGLRATENDGSLSEQLNMSQPMFLNFKKHKVNIYAASHTKVDHIFGRAWAVGVFNTETAAIQKFDLHFPTSTHGALSRFFCFWTGELNIHILNVSTTNAFLKVAHTWFGTDSGIARTATLESNGTMIIPPNEQMTLCVPYYSEVPLRCVKGSDRNSAGLGSLFTQAVGRTISNRVQIFVSFRCPNFFFPLPAPREATSRSILERVDEANAEELEAVLEARTPDAPLRLKFNPEDPLKQLREAAKAYFNIMHSDE

SEQ ID NO: 4 - protein, 296 aa, Ljungan virus M1146
  VP1
GMSSWGSQMDLIDSLDNPEEIQDSEEPESSNVEAAQGQEAATAVGLRATENDGSLTEQVNVAQPMFLNFKQHKVDIYSTSHTKVDHIFGRAWRFQITNLDNSSIGRFTIPFPTTTHGSLARFFAYWTGELNIHVINISTTNAFLKVAHTWFGTTSGIARTGSLESNGVMIIPPNEQMTFCVPYYSEAPLRTVKGTGLSAGLGTFFYQAVGRTIQNRMEIFVSLRCPNFFFPVPAPHEASARSTLARIDTATEEELNAIMSSAEPDTPLRIGKPPEDPLKQLREAAKTYFKIYHDKD

SEQ ID NO: 5 - protein, 296 aa, Ljungan virus 64-7855
  VP1
GEHSWGSQMDLLDSLDNPDEIQDSEEPESSNVEAAQGQDAAKAVGLRATENDGSLTEQINMAQPMFLNYKQHNVDIFSASHTKVDHIFGRAWRHTITNIDNANVGRFTIPFPTTTHGALSRFFAYWTGELNIHVLNISNTNAFMKVAHTWFGTDSGIARTGSLESNGVMIIPPGEQMTMCVPFYSEAPLRTVKGTGASAGLGVFFYQCVGRSVQNRLELFISLRCPNFFFPVPAPHEASSRDVLARLNDATEEELEAVLNARDPDEPLRIGRAPEDPLKQLREAAKAYFKVYHDIE

SEQ ID NO: 6 - protein, 297 aa, Ljungan virus 145SL
  VP1
GLHSWGSEMDLLDSLDNPEEIQDMEEPESENVEAAQGEEAATAVGLRATENDGSLSEQQNMAQPMFLNFKQHRVDIYSASHTKVDHIFGRAWAVGIFNVTNANISKFDLNFPTTTHGALCRFFCFWTGELNLHILNISSSNAPVKVAHTWFGTDSGIARTATLESNGVIIIPPNEQMTLCIPYYSEAPLRCVKGPHSAGAGLGSIFTQCIGNSVNNRIQIFVSFRCPNFFFPLPAPHEASSRSILQRISTASADELEAVLDAKTPDAPVRLCYQPEDPLRQLREAAKAHFNIMHNDE

SEQ ID NO: 7 - protein, 297 aa, Ljungan virus 174F
  VP1
GLHSWGSEMDLVDSLDNPEEIQDNEEIQTQNVEAAQGEEAATEVGLRATENDGSLSEQLNMSQPMFLNFKKHKVNIYAASHTKVDHIFGRAWAVGVFNTETAAIQKFDLHFPTSTHGALSRFFCFWTGELNIHILNVSTTNAFLKVAHTWFGTDSGIARTATLESNGTMIIPPNEQMTLCVPYYSEVPLRCVKGSDRNSAGLGSLFTQAVGRTISNRVQIFVSFRCPNFFFPLPAPREATSRSILERVDEANAEELEAVLEARTPDAPLRLKFNPEDPLKQLREAAKAYFNIMHSDE

SEQ ID NO: 8 - protein, 297 aa, Ljungan virus; identical to SEQ ID NO: 3
  NP_705876.1

SEQ ID NO: 9 - DNA, 35 bp, Artificial Sequence
  sense primer
ttattaggatcggtttgtactcatggggttcagaa

SEQ ID NO: 10 - DNA, 50 bp, Artificial Sequence
  antisense primer
taataagcggccgcttatgggttggtttccacgtcaccacattggttcaa

(19) United States
(12) Patent Application Publication
(10) Pub. No.: US 2022/0273624 A1
(43) Pub. Date: Sep. 1, 2022

(54) NOVEL ANTIVIRAL THERAPIES

(71) Applicant: NIKLOVIR AB, Stockholm (SE)

(72) Inventor: Bo NIKLASSON, Stockholm (SE)

(21) Appl. No.: 17/616,561

(22) PCT Filed: Apr. 22, 2020
(86) PCT No.: PCT/SE2020/050405

§ 371 Date: Dec. 3, 2021

(30) Foreign Application Priority Data
Jun. 28, 2019 (SE) 1950810-0

(51) Int. Cl. A61K 31/4245; A61K 31/501; A61K 31/09; A61K 31/536; A61K 31/138; A61K 31/13; A61K 31/137; A61K 31/404; A61K 31/496; A61K 31/7056; A61K 38/16; A61P 31/12

(52) U.S. Cl. CPC A61K 31/4245; A61K 31/501; A61K 31/09; A61K 31/536; A61K 31/138; A61K 31/13; A61K 31/137; A61K 31/404; A61K 31/496; A61K 31/7056; A61K 38/162; A61P 31/12

(57) ABSTRACT

The present invention relates to a combination of a picornaviridae capsid binding anti-viral agent and at least one further anti-viral agent for use in a method for treatment of a picornavirales infection in a mammalian subject comprising administering said combination to said mammalian subject. It further relates to an anti-viral agent for use in a method for 5 treatment of a picornavirales infection in a mammalian subject comprising administering said anti-viral agent to said mammalian subject, wherein the picornavirales infection is caused by a virus causing in vivo-expression of a VP1-protein detectable in infected tissue through immunohistochemical staining using an antibody raised against the VP1-protein of Ljungan virus, while said virus is not detectable in said infected tissue using a PCR assay 10 adapted for detection of Ljungan virus, as well as a pharmaceutical composition comprising Ljungan virus VP1 protein and a pharmaceutically acceptable adjuvant.

### FIELD OF THE INVENTION

The present invention relates to the field of pharmaceuticals, and in particular to new combinations of antiviral agents useful in treatment of picornavirales infections and the use of anti-viral agents in methods of treatment of infections by a previously uncharacterized virus.

### BACKGROUND OF THE INVENTION

In humans, the virus family Picornaviridae causes the widest range of diseases of all virus families. Infection with various picornaviruses may be asymptomatic or may cause clinical syndromes such as the common cold, febrile rash illnesses, conjunctivitis, hepatitis, myositis and myocarditis. Many picornaviruses have also been shown to have the ability to infect the central nervous system and cause various neurological symptoms, such as meningo-encephalitis and myelitis. Among these viruses, poliovirus is best known, while other non-polio picornaviruses account for approximately one half of aseptic meningitis cases in children.

Ljungan virus (LV) is a member of the Parechovirus genus in the family Picornaviridae. LV infected bank voles in captivity develop several different pathological signs and symptoms including myocarditis, diabetes, encephalitis and stereotypic behavior.

Theiler's murine encephalomyelitis virus, a close relative to LV, cause progressive disability by irreversible CNS tissue injury in mice. This provides an example of how a picornavirus can cause inflammation, demyelination and neural damage. Memory impairment that correlates with the hippocampal injury is seen in this animal model.

Deformed wing virus (DWV) is an Iflavirus (family iflaviridae order picornavirales). The virus infect honey bees (Apis mellifera) causing damaged stubby, useless wings, shortened, rounded abdomens and paralysis of the legs and wings. Artificial infection of bees with this virus result in impaired associative learning and memory formation.

It has been suggested in EP 1 596 859 that Ljungan virus can cause various diseases in humans and animals, such as myocarditis, cardiomyopathy, diabetes, MS, Chronic Fatigue Syndrome, ALS and others.

EP 1 596 859 also suggests the use of pleconaril to treat such diseases. Pleconaril is an orally bioavailable and systemically acting small-molecule drug originally developed for treatment of diseases associated with picornavirus infections (Pevear et al., 1999).

It has furthermore been shown that onset of diabetes in diabetes-prone Biobreeding rats infected with Ljungan virus can be significantly delayed by treatment with a combination of the antiviral drugs pleconaril and ribavirin, indicating that the presence of Ljungan virus can be involved in the pathogenesis of diabetes in these animal models (Holmberg et al., 2009).

The combination of pleconaril and (3,4-dimethoxyphenyl)-N-[(4-fluorophenyl)methyl]-2,5-dimethyl-pyrazolo[1,5-a]pyrimidin-7-amine has been suggested as an antiviral treatment for diabetes prevention in rats (Niklasson et al., Diabetes Prevention Through Antiviral Treatment in Biobreeding Rats, 2016)

### SUMMARY OF THE INVENTION

In a first aspect, the present invention relates to a combination of a picornaviridae capsid binding anti-viral agent and at least one further anti-viral agent for use in a method for treatment of a picornavirales infection in a mammalian subject comprising administering said combination to said mammalian subject, with the proviso that when the picornaviridae capsid binding anti-viral agent is pleconaril then the further anti-viral agent is not solely ribavirin or solely (3,4-dimethoxyphenyl)-N-[(4-fluorophenyl)methyl]-2,5-dimethyl-pyrazolo[1,5-a]pyrimidin-7-amine.

In one embodiment, the picornaviridae capsid-binding anti-viral agent is selected from the group consisting of pleconaril, vapendavir, and pocapavir.

In one embodiment, at least one further anti-viral agent is selected from the group consisting of efavirenz, fluoxetine, amantadine, formoterol, carvedilol, itraconazole, and kinase inhibitors.

In one embodiment, the combination comprises at least pleconaril and efavirenz.

In one embodiment, the combination comprises pleconaril, efavirenz and ribavirin.

In one embodiment, the picornavirales infection is a picornaviridae infection, such as a parechovirus infection.

In one embodiment, the infection is caused by a virus causing in vivo-expression of a VP1-protein detectable in infected tissue through immunohistochemical staining using an antibody raised against the VP1-protein of Ljungan virus, while said virus is not detectable in said infected tissue using an RT-PCR assay adapted for detection of Ljungan virus.

In one aspect, the invention relates to an anti-viral agent for use in a method for treatment of a picornavirales infection in a mammalian subject comprising administering said anti-viral agent to said mammalian subject, wherein the picornavirales infection is caused by a virus causing in vivo-expression of a VP1-protein detectable in infected tissue through immunohistochemical staining using an antibody raised against the VP1-protein of Ljungan virus, while said virus is not detectable in said infected tissue using a PCR assay adapted for detection of Ljungan virus.

In one embodiment, the anti-viral agent is selected from the group consisting of picornaviridae capsid binding anti-viral agents, including pleconaril, vapendavir, and pocapavir; efavirenz; fluoxetine, amantadine, formoterol, carvedilol, itraconazole, ribavirin, kinase inhibitors, and any combination thereof.

In one embodiment, the mammalian subject is of a species selected from the group consisting of human, cat, dog, pig, horse, cow, and camel.

In one embodiment, the picornavirales infection is a picornaviridae infection, such as a parechovirus infection.

In one embodiment, the picornavirales infection is an infection of the central nervous system, pancreas, liver, endocrine tissue, vascular tissue and/or muscle tissue of a subject.

In one embodiment, the infection is of the central nervous system and the subject suffers from a neurological disease, such as Alzheimer disease, Multiple Sclerosis, Amyotrophic Lateral Sclerosis, Chronic Fatigue Syndrome, frontal lobe dementia, or Parkinson's disease.

In one embodiment, the infection is of the pancreas and the subject suffers from Type 1 Diabetes or Type 2 Diabetes.

In one embodiment, the infection is of the liver and the subject suffers from hepatitis.

In one embodiment, the infection is of endocrine tissue and the subject suffers from dysregulation of endocrine function.

In one embodiment, the infection is of myocardium and the subject suffers from heart muscle disease.

In one aspect, the invention relates to a combination of a picornaviridae capsid binding anti-viral agent and at least one further anti-viral agent for use in the manufacture of a pharmaceutical composition for use in a method for treatment of a picornavirales infection in a subject according to invention.

In one aspect, the invention relates to an anti-viral agent for use in the manufacture of a pharmaceutical composition for use in a method for treatment of a picornavirales infection in a subject according to the invention.

In one aspect, the invention relates to a method for treatment of a picornavirales infection according to the above.

In one aspect, the invention relates to a pharmaceutical composition comprising Ljungan virus VP1 protein and a pharmaceutically acceptable adjuvant.

In one aspect, the invention relates to a Ljungan virus VP1 protein, or the pharmaceutical composition according to the above, for use in a method of treatment or prevention of an infection, in a mammalian subject, by a virus causing in vivo-expression of a protein detectable in infected tissue through immunohistochemical staining using an antibody raised against the Ljungan virus VP1 protein, while said virus is not detectable in said infected tissue using a PCR assay adapted for detection of Ljungan virus.

In one aspect, the invention relates to a Ljungan virus VP1 protein, or the pharmaceutical composition according to the above, for use according to the above, wherein the mammalian subject is of a species selected from the group consisting of human, cat, dog, pig, horse, cow, and camel.

### FIGURES

FIG. 1: Microphotograph of formalin-fixed tissue from a 79-year-old patient diagnosed with Alzheimer's disease staining positive using rabbit anti-Ljungan virus VP1 antibodies. Dark stain visualizes presence of viral antigen in neurons, astrocytes and glial cells. In FIG. 1A is shown an amyloid/neuritic plaque (marked with an arrow) staining positive in the glial compartment and in dystrophic neurites. FIG. 1B shows positive staining in neurons (marked with an arrow).

FIG. 2: This figure illustrates the time to onset of diabetes in the seven different treatment groups. Control A (n=9), Pleconaril B (n=6), Pleconaril-Ribavirin C (n=7), Pleconaril—Efavirenz D (n=5), Pleconaril—Efavirenz—Ribavirin E half dose (n=16), Pleconaril—Efavirenz—Ribavirin F full dose (n=12), and Pleconaril-Carvedilol G (n=4).

FIG. 3: IHC-stained tissues in bees infected with DWV (A), and IHC-stained tissues in bees from a colony without DWV virus infection (B).

### DEFINITIONS

“Ljungan-Like Virus” or “LLV” relates to a virus that fulfils at least the two criteria of
(i) It comprises in its genome a nucleic acid encoding a protein that binds to and can be detected by an antibody raised against the Ljungan virus protein VP1;
(ii) It does not comprise in its genome a nucleic acid amplifiable in a PCR using the oligonucleotides 5′-gCggTCCCACTCTTCACAg-3′ (SEQ ID NO: 1) and 5′-gCCCAgAggCTAgTgTTACCA-3′ SEQ ID NO: 2) as primers.

The taxonomy of the order picornaviruses is based on serotypes, species, genus and family. Nucleic acid based diagnostic tests for detection of picornaviruses in clinical specimens target the highly conserved sites of the 5′untranslated region (5′-UTR) allowing diagnosis of different members within a genus in the picornavirus family. The sequence of the 5′UTR PCR product does however not correlate with serotype and does consequently not provide serotype identification. Picornavirus VP1 protein contains a number of neutralizing domains and therefor VP1 sequence correlates with neutralization. It is well known that sequence of the VP 1 capsid gene correlates with the serotype and is therefore used for detection and determination of a species within the genus.
1. The present finding that tissue from patients with disease investigated with IHC is positive using antibodies raised against Ljungan virus VP1 clearly shows that the virus detected has a relationship with Ljungan virus.
2. The Ljungan virus VP1 antiserum has been tested and found positive to all known Ljungan viruses and negative to all other known picornaviruses including those known to be related to LV (Tolf, 2008). The one exception recognized is Deformed Wing Virus of bees (Iflavirus in the iflaviridae family of the order picornavirales) showing reactivity to Ljungan virus VP1 antiserum.
3. The fact that clinical specimens show presence of virus in significant amounts measured by IHC staining and at the same time are negative using a sensitive and specific PCR targeting the 5″UTR show that the virus detected is a not yet recognized virus (or group of viruses) distinct from Ljungan virus. This virus (or group) is herein called Ljungan-like viruses. The reactivity in IHC using VP 1 antisera and at the same time negative by 5′UTR PCR thus defines the term Ljungan-like virus.

Ljungan virus protein VP1 is a capsid protein encoded by Ljungan virus. At least six variants of VP1 from different strains of Ljungan virus have been reported.

Strain 87-012
(SEQ ID NO: 3)
GLHSWGSEMDLVDSLDNPDEIQDNEEIQTQNVEAAQGEEAATEVGLRATENDGSLSEQLNMSQPMFL
NFKKHKVNIYAASHTKVDHIFGRAWAVGVFNTETAAIQKFDLHFPTSTHGALSRFFCFWTGELNIHILNVS
TTNAFLKVAHTWFGTDSGIARTATLESNGTMIIPPNEQMTLCVPYYSEVPLRCVKGSDRNSAGLGSLFTQ
AVGRTISNRVQIFVSFRCPNFFFPLPAPREATSRSILERVDEANAEELEAVLEARTPDAPLRLKFNPEDPLKQ
LREAAKAYFNIMHSDE
Strain M1146
(SEQ ID NO: 4)
GMSS WGSQMDLIDS LDNPEEIQDS EEPESSNVEA AQGQEAATAV GLRATENDGS LTEQVNVAQP
MFLNFKQHKV DIYSTSHTKV DHIFGRAWRF QITNLDNSSI GRFTIPFPTT THGSLARFFA
YWTGELNIHV INISTTNAFL KVAHTWFGTT SGIARTGSLE SNGVMIIPPN EQMTFCVPYY
SEAPLRTVKG TGLSAGLGTF FYQAVGRTIQ NRMEIFVSLR CPNFFFPVPA PHEASARSTL ARIDTATEEE
LNAIMSSAEP DTPLRIGKPP EDPLKQLREA AKTYFKIYHD KD
Strain 64-7855
(SEQ ID NO: 5)
GEHS WGSQMDLLDS LDNPDEIQDS EEPESSNVEA AQGQDAAKAV GLRATENDGS LTEQINMAQP
MFLNYKQHNV DIFSASHTKV DHIFGRAWRH TITNIDNANV GRFTIPFPTT THGALSRFFA
YWTGELNIHV LNISNTNAFM KVAHTWFGTD SGIARTGSLE SNGVMIIPPG EQMTMCVPFY
SEAPLRTVKG TGASAGLGVF FYQCVGRSVQ NRLELFISLR CPNFFFPVPA PHEASSRDVL
ARLNDATEEE LEAVLNARDP DEPLRIGRAP EDPLKQLREA AKAYFKVYHD IE
Strain 145SL
(SEQ ID NO: 6)
GLHSWGSEMDLLDSLDNPEEIQDMEEPESENVEAAQGEEAATAVGLRATENDGSLSEQQNMAQPMFL
NFKQHRVDIYSASHTKVDHIFGRAWAVGIFNVTNANISKFDLNFPTTTHGALCRFFCFWTGELNLHILNIS
SSNAPVKVAHTWFGTDSGIARTATLESNGVIIIPPNEQMTLCIPYYSEAPLRCVKGPHSAGAGLGSIFTQCI
GNSVNNRIQIFVSFRCPNFFFPLPAPHEASSRSILQRISTASADELEAVLDAKTPDAPVRLCYQPEDPLRQLR
EAAKAHFNIMHNDE
Strain 174F
(SEQ ID NO: 7)
GLHSWGSEMDLVDSLDNPEEIQDNEEIQTQNVEAAQGEEAATEVGLRATENDGSLSEQLNMSQPMFL
NFKKHKVNIYAASHTKVDHIFGRAWAVGVFNTETAAIQKFDLHFPTSTHGALSRFFCFWTGELNIHILNVS
TTNAFLKVAHTWFGTDSGIARTATLESNGTMIIPPNEQMTLCVPYYSEVPLRCVKGSDRNSAGLGSLFTQ
AVGRTISNRVQIFVSFRCPNFFFPLPAPREATSRSILERVDEANAEELEAVLEARTPDAPLRLKFNPEDPLKQ
LREAAKAYFNIMHSDE
NCBI Reference Sequence: NP_705876.1
(SEQ ID NO: 8)
GLHSWGSEMDLVDSLDNPDEIQDNEEIQTQNVEAAQGEEAATEVGLRATENDGSLSEQLNMSQPMFL
NFKKHKVNIYAASHTKVDHIFGRAWAVGVFNTETAAIQKFDLHFPTSTHGALSRFFCFWTGELNIHILNVS
TTNAFLKVAHTWFGTDSGIARTATLESNGTMIIPPNEQMTLCVPYYSEVPLRCVKGSDRNSAGLGSLFTQ
AVGRTISNRVQIFVSFRCPNFFFPLPAPREATSRSILERVDEANAEELEAVLEARTPDAPLRLKFNPEDPLKQ
LREAAKAYFNIMHSDE

The term “Ljungan virus protein VP1” includes proteins having the above described amino acid sequences, as well as proteins having 50%, such as 60%, 70%, 75%, 80%, 85%, 90%, 95%, 96%, 97%, 98%, or 99%, sequence identity with SEQ ID NO: 3 and showing specific binding to an antibody raised against a protein of SEQ ID NO: 3.

The terms “sequence identity” may indicate a quantitative measure of the degree of identity between two amino acid sequences or two nucleic acids (DNA or RNA) of equal length. When the two sequences to be compared are not of equal length, they are aligned to give the best possible fit, by allowing the insertion of gaps or, alternatively, truncation at the ends of the polypeptide sequences or nucleotide sequences. The “sequence identity” may be presented as percent number, such as at least 40, 50%, 55,%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% amino acid sequence identity of the entire length, when compared and aligned for maximum correspondence, as measured using a sequence comparison algorithm or by visual inspection.

The sequence identity of the polypeptides of the invention can be calculated as (Nref−Ndif)100/Nref, wherein Ndif is the total number of non-identical residues in the two sequences when aligned and wherein Nref is the number of residues in one of the sequences. The sequence identity between one or more sequence may also be based on alignments using the clustalW or ClustalX software. In one embodiment of the invention, alignment is performed with the sequence alignment method ClustalX version 2 with default parameters. The parameter set preferably used are for pairwise alignment: Gap open penalty: 10; Gap Extension Penalty: 0.1, for multiple alignment, Gap open penalty is 10 and Gap Extension Penalty is 0.2. Protein Weight matrix is set on Identity. Both Residue-specific and Hydrophobic Penalties are “ON”, Gap separation distance is 4 and End Gap separation is “OFF”, No Use negative matrix and finally the Delay Divergent Cut-off is set to 30%. The Version 2 of ClustalW and ClustalX is described in: Larkin et al. 2007, Clustal W and Clustal X version 2.0. Bioinformatics, 23:2947-2948.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: Proline; group 4: Phenylalanine, Tyrosine, Tryptophan; Group 5: Aspartate, Glutamate, Asparagine, and Glutamine.

In some aspects, the amino acid substantial identity exists over a polypeptide sequences length of 10, 15, 20, 25, 30, 35, 40, 45, 50, 55, 60, 65, 70, 75, 80, 85, 90, 95, 100, 110, 120, 130, 140, 150, 160, 170, 180, 190, 200, 250, 300 amino acids in the polypeptide with a “sequence identity” as defined above.

An “anti-viral agent” is a compound having anti-viral activity. This includes all compounds classified in in class J05 and below in the Anatomical Therapeutic Chemical Classification (ATC) system maintained by the World Health Organization Collaborating Centre for Drug Statistics Methodology (https://www.whocc.no/atc_ddd_index/). The ATC classification also includes a Defined Daily Dose (DDD) that is considered therapeutically effective. Unless otherwise indicated herein, an antiviral agent for use according to the invention is to be administered according to the Defined Daily Dose for that agent as provided in the ATC register.

### DETAILED DESCRIPTION OF THE INVENTION

The present inventor has found that a virus apparently closely related to, but clearly distinguishable from, Ljungan virus is likely involved in a range of human diseases. This Ljungan-like virus (LLV) is detectable in infected tissue through immunohistochemical staining using an antibody raised against the VP1-protein of Ljungan virus. The VP1 protein of LLV thus shares immunogenic epitopes with VP1 protein from Ljungan virus. However, LLV is not detectable in said infected tissue using an RT-PCR assay adapted for detection of Ljungan virus (Donoso Mantke et al., 2007), and is thus clearly distinguishable from Ljungan virus.

The present inventor has also unexpectedly found that infections by the Ljungan-like virus can be treated with a combination of anti-viral agents, including capsid-binding agents and at least one further anti-viral agent. This is further described in Example 1.

It has also been found that a protein sharing immunogenic epitopes with the Ljungan virus protein VP1 can be detected in brain tissue from deceased Alzheimer disease patients, but not in age-matched controls that have not been diagnosed with AD. Similar findings have been made in patients affected by Parkinson's Disease, Frontal Lobe Dementia, Multiple Sclerosis and Amyotrophic Lateral Sclerosis. This is further described in Example 2.

As discussed above, it has been suggested that Ljungan virus can cause various diseases in humans and animals, such as Alzheimer disease, myocarditis, Type 1 diabetes, Multiple Sclerosis, Chronic Fatigue Syndrome, Amyotrophic Lateral Sclerosis. It has now been found that a number of human subjects affected by these diseases where in fact not infected by Ljungan virus. This is further described in Example 3.

It has furthermore been found that therapy using combinations of anti-viral agents according to the present invention can alleviate symptoms associated with e.g. Alzheimer disease. This is further described in Example 4.

It has furthermore been found that therapy using certain combinations of anti-viral agents according to the present invention can be used in treatment, prevention, delay of onset, or alleviation of symptoms of Type 1 Diabetes. This is described in Example 5. Analysis of a pancreas sample from a human organ donor with recent onset of T1D and pancreas samples from BB rats support a link between infection by Ljungan or Ljungan-like virus and T1D.

This is consistent with a theory that chronic infection by the Ljungan-like virus (LLV) cause or complicate a range of human diseases and that treatment of the underlying viral infection with the anti-viral agents and novel anti-viral agent combinations according to the present invention can significantly alleviate the symptoms of such disease, including but not limited to Alzheimer disease, Type 1 diabetes, Multiple Sclerosis, Chronic Fatigue Syndrome, frontal lobe dementia, Amyotrophic Lateral Sclerosis, Parkinson's disease, Type 1 Diabetes, hepatitis, dysregulation of endocrine function, and heart muscle disease.

Thus, in a first aspect, the present invention relates to a combination of a picornaviridae capsid binding anti-viral agent and at least one further anti-viral agent for use in a method for treatment of a picornavirales infection in a mammalian subject comprising administering said combination to said mammalian subject.

The following compounds are currently preferred as picornaviridae capsid binding anti-viral agents.

Pleconaril

The compound 3-[3,5-dimethyl-4-[3-(3-methylisoxazol-5-yl)propoxy]phenyl]-5-(trifluoro-methyl)-1,2,4-oxadiazole (generic name: pleconaril, brand name: Picovir®), has the structural formula:

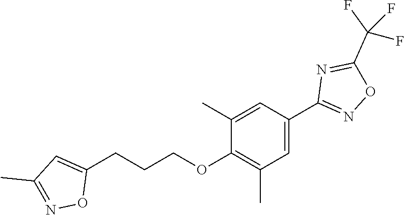 [SMILES: Cc1cc(CCCOc2c(C)cc(-c3noc(C(F)(F)F)n3)cc2C)on1]

Compositions comprising pleconaril and derivatives thereof, as well as their use as an antiviral agent, are disclosed e.g. in U.S. Pat. No. 5,464,848. Pleconaril was originally developed for prevention of asthma exacerbations and common cold symptoms in asthmatic subjects exposed to picornavirus respiratory infections.

Dose in humans 600 mg/day (min 400. max 800)

Vapendavir

The compound 3-ethoxy-6-[2-[1-(6-methylpyridazin-3-yl)-4-piperidyl]ethoxy]-1,2-benz-oxazole, also referred to as vapendavir or BTA798, of structural formula:

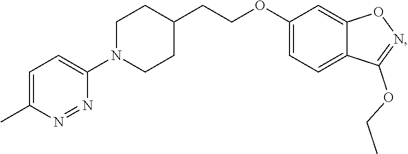 [SMILES: CCOc1noc2cc(OCCC3CCN(c4ccc(C)nn4)CC3)ccc12]

is described in U.S. Pat. No. 7,166,604 which also describes a method for preparing said compound and its use as an antiviral agent for the treatment of picornaviral infections in a mammal.

Dose in humans 900 mg/day (min 600. Max 1200)

Pocapavir

The compound 2-chloro-1-[[4-[(2,6-dichlorophenoxy)methyl]phenyl]methoxy]-4-methoxybenzene, also referred to as Pocapavir and Sch 48973 and V-073, of structural formula is described in European patent No. 0519702 which also describes a method for preparing said compound and its use as an antiviral agent for the treatment of picornaviral infections in a mammal. The drug is a potent and selective antienterovirus agent that is under clinical investigation to treat neonatal enterovirus sepsis, poliovirus infection etc

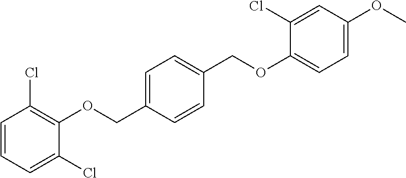 [SMILES: COc1ccc(OCc2ccc(COc3c(Cl)cccc3Cl)cc2)c(Cl)c1]

Dose humans 1600 mg/day (min 1000. max 2000).

The following anti-viral agents are presently preferred as further anti-viral agents in the pharmaceutical combination according to the invention.

Efavirenz

The compound (4S)-6-chloro-4-(2-cyclopropylethynyl)-4-(trifluoromethyl)-1H-3,1-benz-oxazin-2-one (generic name efavirenz, brand names Sustiva® and Stocrin®) of structural formula

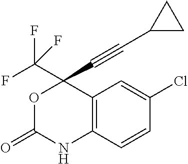 [SMILES: O=C1Nc2ccc(Cl)cc2[C@@](C#CC2CC2)(C(F)(F)F)O1]

is a non-nucleoside reverse transcriptase inhibitor and is presently used as part of highly active antiretroviral therapy (HAART) for the treatment of a human immunodeficiency virus (HIV) type 1.

Dose in humans 600 mg/day (min 400. max 800)

Amantadine

Amantadine is a medicine that is used to treat and prevent infection with influenza (flu) viruses. It also is effective in treating some symptoms of Parkinson's disease.

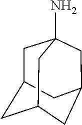 [SMILES: NC12CC3CC(CC(C3)C1)C2]

Dose in humans 300 mg/day (min 100. max 500).

Fluoxetine

Fluoxetine, sold under the brand names Prozac® and Sarafem® among others, is an antidepressant of the selective serotonin reuptake inhibitor (SSRI) class. It is used for the treatment of major depressive disorder, obsessive—compulsive disorder (OCD), bulimia nervosa, panic disorder, and premenstrual dysphoric disorder. It has also been shown to have effect on picornavirus.

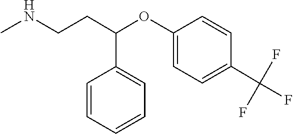 [SMILES: CNCCC(Oc1ccc(C(F)(F)F)cc1)c1ccccc1]

Dose in humans 60 mg/day (min 20. max 80).

Formoterol

Formoterol, N-[2-hydroxy-5-[1-hydroxy-2-[1-(4-methoxyphenyl)propan-2-ylamino]ethyl]phenyl]-formamide, is an adrenergic beta-2 receptor agonist with a prolonged duration of action. It is used to manage asthma and in the treatment of chronic obstructive pulmonary disease. Formoterol has also been shown to inhibit enterovirus replication (Ulferts et al., 2016).

Carvedilol

Carvedilol, 1-(9H-carbazol-4-yloxy)-3-[2-(2-methoxyphenoxy)ethylamino]propan-2-ol, is a non-selective beta blocker indicated in the treatment of mild to moderate congestive heart failure (CHF).

Dose: 37.5 mg/day

Itraconazol

Itraconazole, 2-butan-2-yl-4-[4-[4-[4-[[2-(2,4-dichlorophenyl)-2-(1,2,4-triazol-1-ylmethyl)-1,3-dioxolan-4-yl]methoxy]phenyl]piperazin-1-yl]phenyl]-1,2,4-triazol-3-one, is a triazole antifungal agent that inhibits cytochrome P-450-dependent enzymes required for ergosterol synthesis. It has also been shown to have anti-viral properties (Lee et al., 2017)

Ribavirin

Ribavirin is a synthetic nucleoside analog of ribofuranose with activity against hepatitis C virus and other RNA viruses. Ribavirin is incorporated into viral RNA, thereby inhibiting viral RNA synthesis, inducing viral genome mutations, and inhibiting normal viral replication. The combination of pleconaril and ribavirin as the sole additional active ingredient is not part of the present invention.

Dose: 1,000 mg/day (adult humans)

Kinase Inhibitors

The phosphatidylinositol 4-kinase PI4KIIIβ is critical for mediating viral replication of a number of RNA viruses through the generation of PI4P enriched viral replication platforms. Inhibitors of PI4KIIIβ are thus anti-viral agents useful in the present invention.

(3,4-dimethoxyphenyl)-N-[(4-fluorophenyl)methyl]-2,5-dimethyl-pyrazolo[1,5-a]pyrimidin-7-amine, also termed APO-N039, is one example of an inhibitor of kinase PI4KIIIB which has been shown to have anti-viral effect (Niklasson et al., Diabetes Prevention Through Antiviral Treatment in Biobreeding Rats, 2016). This compound is further described in WO2015110491. The combination of pleconaril and APO-N039 as the sole additional active ingredient is not part of the present invention.

Other exemplary PI4KIIIβ inhibitors are PI4KIIIbeta-IN-10 (CAS No.: 1881233-39-1); PIK-93 (CAS No.: 593960-11-3); PI4KIIIbeta-IN-9 (CAS No.: 1429624-84-9); PI4KIII beta inhibitor 3 (CAS No.: 1245319-54-3); UCB9608 (CAS No.: 1616413-96-7); BF738735 (CAS No.: 1436383-95-7); BQR-695 (CAS No.: 1513879-21-4); T-00127_HEV1 (CAS No.: 900874-91-1) (all commercially available from MedChem Tronica, Sollentuna, Sweden).

In a preferred embodiment, the combination of a picornaviridae capsid binding anti-viral agent and at least one further anti-viral agent comprises at least pleconaril and efavirenz. This combination has shown a surprising synergistic effect in treatment of infections by picornavirales, as disclosed in Examples 1, 4, and 5.

In another preferred embodiment, the combination of a picornaviridae capsid binding anti-viral agent and at least one further anti-viral agent comprises at least pleconaril and carvedilol. This combination has shown a surprising synergistic effect in delay of onset of diabetes in virally infected diabetic prone rats, as disclosed in Example 5.

In another preferred embodiment, the combination of a picornaviridae capsid binding anti-viral agent and at least one further anti-viral agent comprises at least pleconaril, efavirenz and ribavirin. This combination has shown a surprising synergistic effect in delay of onset of diabetes in virally infected diabetic prone rats, as disclosed in Example 5.

In one embodiment, the infection is caused by a Ljungan-like virus, i.e. a virus causing in vivo-expression of a VP1-protein detectable in infected tissue through immunohistochemical staining using an antibody raised against the VP1-protein of Ljungan virus, while said Ljungan-like virus is not detectable in said infected tissue using an RT-PCR assay adapted for detection of Ljungan virus.

In one aspect, the present invention relates to an anti-viral agent for use in a method for treatment of a picornavirales infection in a mammalian subject comprising administering said anti-viral agent to said mammalian subject, wherein the picornavirales infection is caused by a Ljungan-like virus, i.e. a virus causing in vivo-expression of a VP1-protein detectable in infected tissue through immunohistochemical staining using an antibody raised against the VP1-protein of Ljungan virus, while said virus is not detectable in said infected tissue using a PCR assay adapted for detection of Ljungan virus.

In one embodiment, the method of treatment includes a step of identifying a subject as being infected by a Ljungan-like virus, preferably prior to administration of the anti-viral agent. Subjects infected by the Ljungan-like virus can be identified by analysing tissue samples from said patient with both an immunohistochemical assay and a PCR based assay, as disclosed herein. Patients whose samples are positive for Ljungan-like virus, such as positive in the immunohistochemical assay and negative in the PCR-based assay, are considered suitable for treatment with the anti-viral agent, or combination of anti-viral agents, according to the invention. Suitable tissue sample include buffy coat and tissue biopsies such as from muscle, pancreas, liver, endocrine glands, blood vessels, nerves or placenta.

In one embodiment, the anti-viral agent is selected from the group consisting of picornaviridae capsid binding anti-viral agents, including pleconaril, vapendavir, and pocapavir; efavirenz; fluoxetine, amantadine, formoterol, carvedilol, itraconazole, ribavirin, kinase inhibitors, and any combination thereof.

The anti-viral agents and combinations disclosed herein are suitable for treatment of any mammalian subject infected, including but not limited to human, cat, dog, pig, horse, cow, and camel subjects.

In one embodiment, the anti-viral agents and combinations disclosed herein are used for treatment of a picornavirales infection that is an infection of the central nervous system, pancreas, liver, endocrine tissue, vascular tissue kidney, placenta and/or muscle tissue of a subject.

In one specific embodiment, the infection is of the central nervous system and the subject suffers from a neurological disease, including but not limited to Alzheimer disease, Multiple Sclerosis, Amyotrophic Lateral Sclerosis, Chronic Fatigue Syndrome, Parkinson's disease and Frontal Lobe Dementia.

In one specific embodiment, the infection is of the pancreas and the subject suffers from Type 1 Diabetes or Type 2 Diabetes.

In one specific embodiment, the infection is of the liver and the subject suffers from hepatitis.

In one specific embodiment, the infection is of endocrine tissue and the subject suffers from dysregulation of endocrine function.

In one specific embodiment, infection is of myocardium and the subject suffers from heart muscle disease, including but not limited to myocarditis and cardiomyopathy. In one aspect, the present invention relates to a combination of a picornaviridae capsid binding anti-viral agent and at least one further anti-viral agent for use in the manufacture of a pharmaceutical composition for use in a method for treatment of a picornavirales infection in a subject according to the invention as described herein.

In one aspect, the present invention relates to an anti-viral agent for use in the manufacture of a pharmaceutical composition for use in a method for treatment of a picornavirales infection in a subject according to the invention as described herein.

In one aspect, the invention relates to a method for treatment of a picornavirales infection comprising administering the anti-viral agents or combination of anti-viral agents as described herein to a mammalian subject in need thereof, as disclosed herein.

In one aspect, the invention relates to a pharmaceutical composition comprising Ljungan virus VP1 protein and a pharmaceutically acceptable adjuvant. Such compositions are useful in immunisation and vaccination of subjects having, or at risk of acquiring, a picornavirales infection, such as an infection by the Ljungan-like virus as described herein. Vaccination is usually intended to be performed prior to exposure to the picornavirales infection to activate the immune system in the host to elicit an immune response towards the VP1 protein and/or the infectious agent. It is also possibly to administer the VP1 protein or pharmaceutical composition after exposure and after an infection has been detected. The virus may be able to hide inside the host's cells and evade the host's immune system. A vaccination may then trigger an immune response that serves to combat the infection.

Pharmaceutically acceptable adjuvants are used to enhance the immunogenic response to the antigen, in this case the Ljungan virus protein VP1, in an immunotherapy, and are well-known to the skilled person. Suitable adjuvants include an aluminium salt such as aluminium hydroxide gel (alum) or aluminium phosphate, but may also be a salt of calcium, iron or zinc, or may be an insoluble suspension of acylated tyrosine, or acylated sugars, cationically or anionically derivatised polysaccharides, or polyphosphazenes. In the compositions of the inventions it is preferred that the adjuvant composition induces a preferential TH1 response. Suitable adjuvant systems include, for example, a combination of monophosphoryl lipid A or derivative thereof, preferably 3-de-O-acylated monophosphoryl lipid A (3D-MPL) together with an aluminium salt. An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO 96/33739. A particularly potent adjuvant formulation involving QS21, 3D-MPL and tocopherol in an oil in water emulsion is described in WO 95/17210.

In one aspect, the invention relates to a method of treatment or prevention of an infection, in a mammalian subject, by a virus causing in vivo-expression of a protein detectable in infected tissue through immunohistochemical staining using an antibody raised against the Ljungan virus VP1 protein, while said virus is not detectable in said infected tissue using a PCR assay adapted for detection of Ljungan virus, said method comprising administering a Ljungan virus protein VP1 or a pharmaceutical composition comprising Ljungan virus VP1 protein and a pharmaceutically acceptable adjuvant to a mammalian subject in need thereof.

In a further aspect, the invention relates to treatment of disease, prevention of disease, delay of onset of disease, or amelioration of symptoms of a disease, that may be caused or complicated by a picornavirales infection, such as an infection of Ljungan-like virus or Ljungan virus. Such prevention or amelioration utilizes the anti-viral agents or combination of anti-viral agents as discussed herein as antiviral therapies, or the Ljungan VP1 protein in immunotherapy methods, as generally disclosed herein.

Thus, in one embodiment the invention relates to prevention, delay of onset, or amelioration of a neurological disease caused or complicated by a picornavirales infection. In embodiments, the neurological disease is selected from Alzheimer disease, Multiple Sclerosis, Amyotrophic Lateral Sclerosis, Chronic Fatigue Syndrome, Parkinson's disease and Frontal Lobe Dementia.

In one embodiment, the invention relates to prevention, delay of onset, or amelioration of Type 1 Diabetes, Type 2 Diabetes, hepatitis, dysregulation of endocrine function, myocarditis or cardiomyopathy.

In one specific embodiment, the invention relates to prevention or delay of onset of Type 1 Diabetes by administration of a combination of pleconaril and efavirenz, and optionally ribavirin, to a subject having a picornavirales infection, such as an infection of Ljungan-like virus or Ljungan virus.

In certain embodiments, the mammalian subject is of a species selected from the group consisting of human, cat, dog, pig, horse, cow, and camel.

In certain embodiments, when the picornaviridae capsid binding anti-viral agent is pleconaril then ribavirin is not a further anti-viral agent. In certain embodiments when the picornaviridae capsid binding anti-viral agent is pleconaril and ribavirin is a further anti-viral agent, then the mammalian subject is not a rat or a human.

In certain embodiments, when the picornaviridae capsid binding anti-viral agent is pleconaril then (3,4-dimethoxyphenyl)-N-[(4-fluorophenyl)methyl]-2,5-dimethyl-pyrazolo[1,5-a]pyrimidin-7-amine is not a further anti-viral agent. In certain embodiments when the picornaviridae capsid binding anti-viral agent is pleconaril and (3,4-dimethoxyphenyl)-N-[(4-fluorophenyl)methyl]-2,5-dimethyl-pyrazolo[1,5-a]pyrimidin-7-amine is a further anti-viral agent, then the mammalian subject is not a rat or a human.

The contents of all documents, including journal articles, patents, and patent applications, cited herein are expressly incorporated herein by reference.

### EXAMPLES

The examples below are intended to further illustrate the present invention and are not intended to limit the scope of the present invention, which is that of the appended claims.

### Example 1: Treatment of Biobreeding Rats with a Combination of Pleconaril and Efavirenz

The Bio Breeding (BB) rat originates from a Canadian colony of outbred Wistar rats spontaneously developing hyperglycemia that quickly progresses to fatal diabetic ketoacidosis unless treated with exogenous insulin. The fact that all diabetes prone (DP) BB rats develop diabetes in a narrow time frame provides an excellent opportunity to study the impact of treatment prior to diabetes onset. The BB rat is one of the most engaged animal models for the study on β-cell destruction and onset of type1 diabetes (T1D).

The finding of a picornavirus named Ljunganvirus (LV) present in Scandinavian vole and lemming populations and the association of this virus with diabetes in these animals have been reported earlier (Niklasson et al., 2003). The finding of LV antigen was reported in β-cells of diabetic BB rat. LV antigen was also detected in nerve tissue, muscle tissue and in the endothelial cells of blood vessel.

DP-BB rats were obtained from the breeding colony maintained at the Karolinska Institute (Solna, Sweden). The animals were housed under Specific Pathogen Free conditions in a temperature- and humidity-controlled room with 12 hour light dark cycles. They were fed R36 diet and water ad libitum. Animals of both genders were used. The overall incidence of diabetes among the DP rats used in the colony study is 100% with 60 days as the mean age of onset.

Antiviral agents (pleconaril and efavirenz) were administered to the animals in a dose of 100 mg/kg and 30 mg/kg body weight for Pleconaril and Efavirenz, respectively. Treatment was given daily using formulation containing the vehicle 0.4% (v/v) Tween 80+2% (v/v) glycerol+15% (w/v) Hydroxypropyl-beta-cyclodextrin.

Four experimental treatment groups consisted of six rats given pleconaril, six given Efavirenz, eight given a combination of both compounds, and nine given the vehicle solution and serving as a control. All four groups began treatment at 45 days of age and treatment was maintained until 60 days of age when animals were sacrificed.

Formalin fixed paraffin embedded pancreas tissue were analyzed by immunohistochemistry. The tissues were fixed in 4% formalin and embedded in paraffin as described previously with minor modifications (Shi et al., 1991), (Shi, 1997). Presence of LV specific antigen was visualized using a polyclonal recombinant LV VP1 raised in rabbits (Tolf, 2008). As control we used serum from a rabbit immunized using the same protocol but with the carrier GST protein only. Tissues from LV-infected and non-infected animals were included as controls. The specificity of the rabbit antibodies were verified by analyzing control specimens generated by mixing infected tissue culture cells with non-infected cells followed by formalin fixation and paraffin imbedding. The specificity of the reaction was also confirmed by blocking the signal with LV antigen in parallel with control antigen.

Pancreas tissue was also analysed using the PCR assay described by Donoso Mantke and co-workers (Donoso Mantke et al., 2007). The results were negative, indicating that the animals were not infected with Ljungan virus.

The results are disclosed in Table 1.

TABLE 1

| Regime | No of animals | Infected | Non-infected |
|---|---|---|---|
| Pleconaril | 6 | 4 | 2 |
| Efavirenz | 6 | 6 | 0 |
| Pleconaril-Efavirenz | 8 | 0 | 8 |
| Control (no antiviral) | 9 | 9 | 0 |

Most notably, pleconaril alone appears to have only a modest effect on clearing the infection, and efavirenz alone appears to have essentially no effect. Contrary to this, all animals that received the combination therapy pleconaril+efavirenz had undetectable amounts of viral antigen in pancreas tissue following treatment. This supports a strong synergistic effect of a combination according to the invention. This synergistic effect is further investigated in Example 5.

### Example 2: Detection of Picornavirus in Brain Tissue of AD, PD, FLD, MS, and ALS Patients and Healthy Controls

Alzheimer's Disease

The first part of this example investigates formalin-fixed post mortem brain tissue from the hippocampus region of 18 Alzheimer Disease (AD) cases and 11 age matched controls using a polyclonal antibody against Ljungan virus capsid protein 1.

Brain samples from 3 AD cases and 2 controls came from the brain bank at the Department of Oncology and Pathology, University of Lund while 15 AD cases and 9 controls came from the Netherlands Brain Bank. Included patients were between 65-85 years of age. The AD specimens came from deceased individuals being diagnosed based on the NINCDS-ADRDA criteria. These criteria require the presence of cognitive impairment and a suspected dementia syndrome confirmed by neuropsychological testing. All AD cases included in this study were histopathologically confirmed by immunohistochemical (IHC) staining positive for hyperphosphorylated tau, alpha-synuclein protein and histochemical positive staining for amyloid with alcalic congo. Control specimens came from age and sex matched patients with no history of cognitive impairment or suspected dementia syndrome. All controls were also tested negative by IHC for hyperphosphorylated tau, alpha-synuclein protein and were histochemically negative with amyloid staining.

Formalin-fixed paraffin-embedded brain tissue from the hippocampus region was sectioned at 5 micrometers and analyzed for presence of LV antigen using IHC previously described, with minor modifications (Shi et al., 1991), (Shi, 1997). Presence of LV specific antigen was visualized using a polyclonal recombinant LV VP1 raised in rabbits (Tolf, 2008). As control we used serum from a rabbit immunized using the same protocol but with the carrier protein only. Tissues from LV-infected and non-infected animals were included as additional controls. The specificity of the rabbit antibodies were verified by analyzing control specimens generated by mixing infected tissue culture cells with non-infected cells followed by formalin fixation and paraffin embedding. The specificity of the reaction was also confirmed by blocking the signal with LV antigen in parallel with control antigen.

LV viral antigen was detected in the hippocampal sections of all 18 cases with AD but in none of the 11 age matched controls. A control serum consisting of normal rabbit was negative in all 18 AD cases and 11 controls. The association of viral presence in AD patients versus controls were found to be significant (p<0.0001 Fischer's exact two sided test).

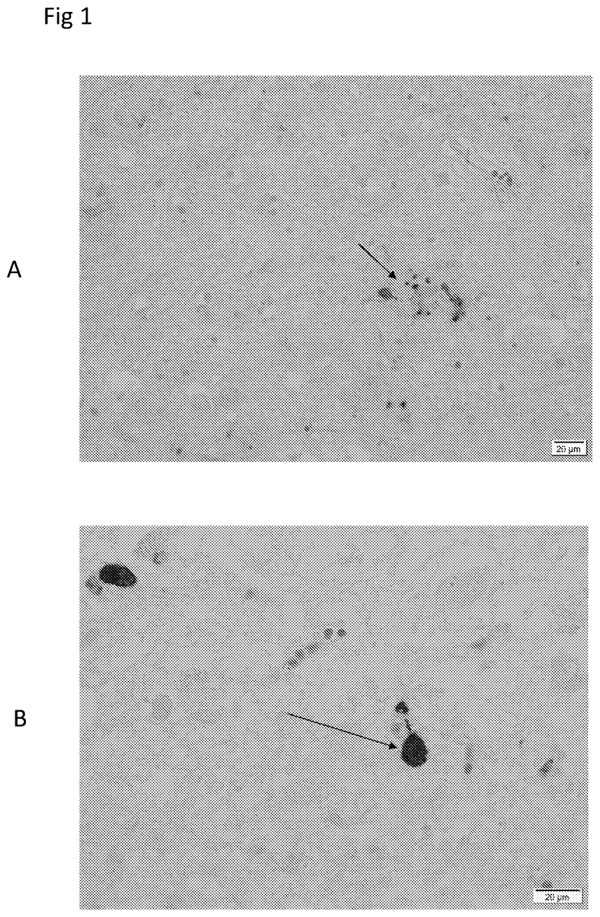

FIG. 1A-B are microphotographs of hippocampus tissue from one 79-year-old AD patient representative of the findings in all AD patients in this study.

The binding of rabbit anti LV VP1 antigen is seen as dark stain showing distinct reactions in most cell types—neurons, astrocytes and microglial cells. An amyloid plaque with positive viral antigen staining in both the glial compartment and in dystrophic neurites is illustrated in FIG. 1A and indicated by an arrow. FIG. 1B is a magnification of a viral antigen positive neuron from the same patient. No antigen positive cells were found in any if the 11 control patients.

Parkinson's Disease, Frontal Lobe Dementia, Multiple Sclerosis, and Amyotrophic Lateral Sclerosis

Samples

Formalin-fixed paraffin-embedded brain tissues from patients with Frontal lobe dementia and Parkinson's disease were received from Netherlands Brain Bank, Netherlands Institute for Neuroscience, Amsterdam, The Netherlands. The specimens from patients with Multiple sclerosis came from department of pathology and department of neurology, Karolinska Institutet and Karolinska University Hospital, Huddinge, Sweden.

Immunohistochemistry

Formalin-fixed paraffin-embedded brain tissue was sectioned at 5 micrometers and analyzed for presence of LV antigen using IHC previously described, with minor modifications (Shi, 1997). Presence of LV specific antigen was visualized using a polyclonal antiserum against recombinant LV immune dominant capsid viral protein 1 (VP1) raised in a rabbit (Tolf, 2008). As control we used serum from a rabbit immunized using the same protocol but with the carrier protein only. Tissues from LV-infected and non-infected animals were included as additional controls. The specificity of the rabbit antibodies were verified by analyzing control specimens generated by mixing LV infected tissue culture cells with non-infected cells followed by formalin fixation and paraffin embedding. The specificity of the reaction was also confirmed by blocking the signal with LV antigen in parallel with control antigen.

Frontal Lobe Dementia (FLD)

Formalin-fixed paraffin-embedded brain tissues from the frontal cortex was analyzed from 10 patients diagnosed with frontal lobe dementia (Picks disease) according the criteria as defined by Mac Kenzie and coworkers (Mackenzie et al., 2010). Three females with mean age were 72 (64-77 years of age) and 7 males with mean age of 68 (57-77 years of age) were analyzed. All cases were diagnosed according the Lund and Manchester Criteria, 1994. Frontal cortex from 10 healthy individuals was also analyzed as controls including 2 females with mean age 46 (32-65 years of age) and 8 males with mean age 53 (38-64 years of age).

LV viral antigen was detected in the frontal cortex of all 10 cases investigated and in 3 of the 10 controls see table 2. The association of viral presence in FD patients versus controls were found to be very significant (p=0.003 Fischer's exact two sided test).

Parkinson's Disease (PD)

All PD cases included had motor symptoms typical of PD before deaths. The diagnosis was confirmed by pathological findings characterized by a selective loss of neurons in the brainstem that produce the neurotransmitters dopamine and noradrenaline. Lewy bodies and Lewy neurites composed of abnormal aggregated proteins, primarily abnormal a-synuclein, were visible inside the affected nerve cells. Formalin-fixed paraffin-embedded Substantia nigra was analyzed from 4 females and 5 males with PD. The mean age of the 4 females was 71 (66-77) and for the 5 males mean years of age 69 (57-78). Substantia nigra was also analyzed from 10 controls including 6 females with mean age of 74 (64-82) and 4 males with mean age of 63 (49-82).

LV viral antigen was detected in the substantia nigra area 7 of 9 cases with PD and on 1 of the 10 healthy controls see table 2. The association of viral presence in PD patients versus controls were found to be very significant (p=0.006 Fischer's exact two sided test).

Multiple Sclerosis (MS)

All MS cases included had presence of signs and symptoms typical of MS before deaths of also typical post mortem pathological brain lesions. Five patients with MS was available including 3 females with mean age of 62 years of age (59, 59, 69 years of age) and 2 men 50 and 72 years of age. Areas with MS typical plaques were selected for analysis. No matched controls were available.

LV viral antigen was detected in the brain tissue with classic white matter plaques in 4 of 5 patients tested. No controls were available for this patient group.

Amyotrophic Lateral Sclerosis (ALS)

Formalin-fixed paraffin-embedded from the brain steam tissue was analyzed from 10 patients diagnosed with sporadic amyotrophic lateral sclerosis according the clinical criteria confirmed by post mortem PAD findings of the central nervous system described by Zarie and co-workers (Zarei et al., 2015). Brain stream tissue from 9 patients without signs and symptoms of neurological disease was included as controls.

The association of viral presence in ALS patients versus controls were found to be very significant (p=0.006 Fischer's exact two sided test).

TABLE 2

| | | FLD | | PD | | | ALS |
|---|---|---|---|---|---|---|---|
| | FLD | control | PD | control | MS | ALS | control |
| IHC | 10 | 3 | 7 | 1 | 4 | 9 | 2 |
| positive | | | | | | | |
| IHC | 0 | 7 | 2 | 9 | 1 | 1 | 7 |
| negative | | | | | | | |
| Total | 10 | 10 | 9 | 10 | 5 | 10 | 9 |

### Example 3: PCR Analysis of Virus Infected Animals and Humans

Buffy coat samples from a number of rodent and human subjects known or suspected to be infected with Ljungan virus were analysed using the PCR assay described by Donoso Mantke and co-workers (Donoso Mantke et al., 2007).

The buffy coat is the fraction of an anticoagulated blood sample that contains most of the white blood cells and platelets following density gradient centrifugation of the blood. After centrifugation, one can distinguish a layer of clear fluid (the plasma), a layer of red fluid containing most of the red blood cells, and a thin layer in between. Composing less than 1% of the total volume of the blood sample, the buffy coat contains most of the white blood cells and platelets.

The results are presented in Table 3.

TABLE 3

| | | | Ljungan virus |
|---|---|---|---|
| | | No of | PCR positive |
| Buffy coat specimens | Species | specimens | (%) |
| LV infected suckling lab mice | Rodent | 12 | 12 (100%) |
| Wild bank voles | Rodent | 15 | 3 (20%) |
| Alzheimer's disease | Human | 11 | 0 (0%) |
| Multiple sclerosis | Human | 10 | 0 (0%) |
| Type 1 diabetes | Human | 22 | 0 (0%) |
| Chronic fatigue syndrome | Human | 8 | 0 (0%) |
| (ME) | | | |
| Amyotrofic lateral sclerosis | Human | 13 | 0 (0%) |
| Myocarditis | Human | 5 | 0 (0%) |

The results indicate that Ljungan virus is not the primary etiological agent in AD, MS, T1D, ME, ALS, or myocarditis.

### Example 4: Treatment of Viral Infections in a Patient Suffering from Alzheimer Disease

An 67-year-old woman with history of decreasing cognitive function was diagnosed with Alzheimer's disease (AD) at the Memory unit at the Geriatric clinic, Karolinska University Hospital Stockholm. The diagnosis was confirmed by analysis of cerebrospinal fluid (beta-amyloid, phosphor tau and tau) and brain magnetic resonance Imaging.

Antiviral treatment with 600 mg Pleconaril and 400 mg Efavirenz daily was initiated 6 months after the diagnosis. The cognitive status of the patient was measured with Mini-Mental State Examination (MMSE) and Alzheimer's Quick Test (AQT) before initiating antiviral treatment and after 4 months of therapy. Both MMSE and AQT are well established and validated instruments for evaluation of cognitive function in AD were MMSE have a maximum score of 30 points decreasing an average of 3 points per year while AQT is a test patients perform on time. AQT time typically increases with a mean of 16 seconds per year. Decreased decline rate (DDR) was used to express the difference between the expected decline rate in a historical control (statistical) population and the clinical change seen in the patient treated with antiviral medicine. The DDR in percent is calculated using the formula DDR=(1−(actual change/expected change))×100. With no decline at all DDR would be 100%, indicating full treatment effect. If patients have a reduced decline as a result of treatment the DDR will be between 1-100%. If patients improve instead of decline the number is expressed as >100%. For patient who decline faster than expected the DDR will show a negative outcome.

### SUMMARY

The patient showed improved cognitive function after receiving antiviral treatment (Table 4). The MMSE measured at the beginning and the end of the 4 months long period of treatment improved by 3 points. AQT improved over the same period from 86 seconds to 53 seconds. This means that DDR for the present patient was >100% for both MMSE and AQT.

TABLE 4

| | MMSE | | | |
|---|---|---|---|---|
| Duration | | | Expected | |
| of | Before | After | outcome with | |
| treatment | treatment | treatment | no treatment | DDR |
| (months) | (points) | (points) | (points) | % |
| | 22 | 25 | 21 | >100 |
| | AQT | | | |
| | Score | Score | Expected | |
| | before | after | score with | |
| | treatment | treatment | no treatment | DDR |
| | (seconds) | (seconds) | (seconds) | % |
| 4 | 86 | 53 | 91 | >100 |

### Example 5: Antiviral Treatment of Diabetic Prone Rats and Comparison to Human T1D

DP BB rats were obtained from the breeding colony maintained at the Karolinska Institutet. The animals were housed under Specific Pathogen Free conditions in a temperature- and humidity-controlled room with 12 hour light dark cycles. They were fed R36 diet and water ad libitum. Animals of both sexes were used. The overall incidence of diabetes among the DP rats used in the colony study is 100% with 60 days as the mean age of onset.

Group A (n=9) is a control group given vehicle only, group B (n=6) Pleconaril alone, group C (n=7) a combination of Pleconaril and Ribavirin, group D (n=5) combination of Pleconaril and Efavirenz, and group E (n=16) and F (n=12) were given a combination of Pleconaril, Ribavirin and Efavirenz. Group E was given half the dose of group F. Group G (n=4) was Pleconaril and Carvedilol. Pleconaril and Carvedilol were administered to the animals in a dose of 100 mg/kg BW and of 40 mg/kg BW, respectively once daily using a 10 mg/ml and 4 mg/ml respectively formulation containing the vehicle 0.4% (v/v) Tween 80+2% (v/v) glycerol+15% (w/v) Hydroxypropyl-beta-cyclodextrin. The results from Group A and B have previously been reported as part of a different study (Niklasson et al., Diabetes Prevention Through Antiviral Treatment in Biobreeding Rats, 2016).

Pleconaril, Ribavirin and Efavirenz were administered to the animals in a dose of 100 mg/kg BW, of 100 mg/kg BW and of 60 mg/kg BW, respectively once daily using a 10 mg/ml, 10/mg/ml and 6 mg/ml respectively formulation containing the vehicle 0.4% (v/v) Tween 80+2% (v/v) glycerol+15% (w/v) Hydroxypropyl-beta-cyclodextrin.

Antiviral compounds and the control vehicle were administrated using an oral gavage once daily. Therapy was initiated between 42-48 days of age and maintained for a total of 30 days. All animals were followed until onset of diabetes (blood glucose 270 mg/dL or above and/or ketons) or until 110 days of age which ever occurred first.

Pancreas tissue was collected for tissue transplantation from a 40 years old male involved in a fatal accident. As part of the screening process prior to transplantation it was discovered that the patient suffered from T1D not yet clinically diagnosed. Formalin-fixed paraffin-embedded pancreatic tissue from this patient (H911) was kindly provided by doctor Gun Frisk, University of Uppsala, Uppsala, Sweden and analyzed for presence of LV antigen by IHC. Formalin fixed paraffin embedded pancreas tissue from 64 days old male BB rat with recent onset of T1D was stained in parallel.

The tissues were fixed in 4% formalin and embedded in paraffin as described previously with minor modifications (Shi, 1997). Presence of LV specific antigen was visualized using a polyclonal recombinant LV VP1 raised in rabbits (Tolf, 2008). As control we used serum from a rabbit immunized using the same protocol but with the carrier GST protein only. Tissues from LV-infected and non-infected animals were included as controls. The specificity of the rabbit antibodies were verified by analyzing control specimens generated by mixing infected tissue culture cells with non-infected cells followed by formalin fixation and paraffin imbedding. The specificity of the reaction was also confirmed by blocking the signal with LV antigen in parallel with control antigen.

The mean age of diabetes onset in the control group was 59 (SD 7) days of age and not found statistically different from the group B receiving Pleconaril alone with mean onset day 62 (SD 8) or from group C receiving Pleconaril-Ribavirin with mean 61 days (SD 7) with p=0.5 and p=0.7, respectively, using unpaired two tailed t-test (see table 5). Group D receiving Pleconaril—Efavirenz had mean day of onset 71 (SD 3) being significantly different versus the control group (p=0.007). Group F receiving Pleconaril—Efavirenz—and Ribavirin had mean day of onset 86 (SD 25) being significantly different from the control group (p=0.005). Group E receiving Pleconaril—Efavirenz—and Ribavirin at half the dose of group F also had mean day of onset 73 (SD 9) being significantly different versus the control group (p=0.001).

Group G receiving Pleconaril and Carvedilol had mean day of onset 69 (SD 2), being significantly different versus the control group (p=0,035).

TABLE 5
Animals receiving different therapeutic regimes during a period of 30 days. Mean
day of onset of diabetes and standard deviation (SD) of the mean time of onset
are given as well as statistical analysis (unpaired t-test) of the difference between
animals in one particular treatment group compared with control animals.

| | | | | | Plec-Rib- | Plec-Rib- | Plec- |
|---|---|---|---|---|---|---|---|
| | Control | Plec | Plec-Rib | Plec-Ef | Ef 50% | Ef 100% | Carv |
| | (A) | (B) | (C) | (D) | (E) | (F) | (G) |
| No of animals | 9 | 6 | 7 | 5 | 16 | 12 | 4 |
| Mean diab | 59 (7) | 62 (8) | 61 (7) | 71 (3) | 73 (9) | 86 (25) | 69 (2) |
| onset day (SD) | | | | | | | |
| Onset delay | | NS | NS | p = 0.007 | p = 0.001 | p = 0.005 | P = 0.035 |
| (stat. sign.) | | (p = 0.5) | (p = 0.7) | | | | |

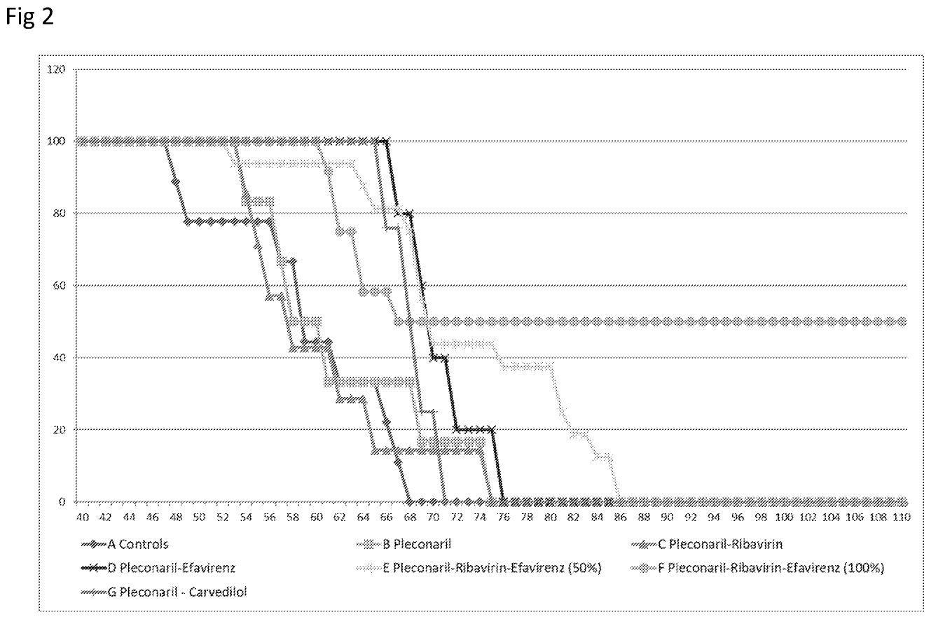

Time to diabetes onset in DP BB rats using the six experimental treatment regime is seen in FIG. 2. In group E a total of 6 rats were non-diabetic at day 110. Three of the 6 animals were not sacrificed at day 110 but at day 200 when all three rats were still non-diabetic. The rats sacrificed at day 200 were all found LV-negative in their pancreas by IHC.

As an illustration of the similarities between T1D in humans and BB rat animal model formalin-fixed paraffin-embedded pancreas tissue from both species collected at the time of diabetes onset was analyzed for presence of LV antigen using IHC (human sample H911 provided by Gun Frisk, University of Uppsala, Uppsala, Sweden). Presence of LV specific virus protein 1 antigen in red was visualized in the pancreas Langerhans islet using a polyclonal recombinant LV VP1 antiserum raised in rabbits (Tolf, 2008). It cannot be determined if it is insulin producing β-cell or glucagon producing α-cell showing LV positive staining. However, the distribution of positive cells suggests that insulin producing cells are infected in both the human and the rat specimen.

It is noted that the experimental groups differ according to the drug and concentration of drugs employed, with the one clearly successful treatment (group F) being more successful in delaying and stopping T1D than a lower dose of the same drugs (group E); surviving animals in group F remained diabetes-free long beyond any typical BB rat (i.e., >200 days) and all without symptoms of diabetes onset. The IHC on BB rats and a human sample with evidence of T1D revealed that LV surface coat protein antibodies were positive in each of the pancreas samples. Each of these concordant lines of evidence come together to point to the likelihood of true effects.

### Example 6: Immunisation of Subjects with VP1

The present example disclose how a subject can be immunised using a capsid protein VP1

The gene sequences encoding the predicted capsid protein VP1 will be amplified using gene specific primers. The VP1 gene including the 2A1 motif, except the two last C-terminal codons (948 bp) will be amplified using the sense primer

(SEQ ID NO: 9)
5′-TTATTAGGATCGGTTTGTACTCATG GGGTTCAGAA-3′

and the antisense primer

(SEQ ID NO: 10)
5′-TAATAAGCGGCCGCTTATGGGTTGGTTTCCACGTCACCACATTGGT
TCAA-3′.

The gene sequences encoding predicted LV capsid proteins VP 1 will be cloned into a bacterial expression vector. The expression vector and recombinant plasmids will be amplified in an Escherichia coli strain and subsequently transferred to E. coli BL-21 (DE3) for protein expression.

Recombinant plasmids and the expression vector without insert (negative control) will be transformed separately into Escherichia coli to express Ljungan virus (LV) VP1 protein as a GST fusion protein or as the GST tag protein alone.

Alternatively, the capsid protein VP1 is produced through peptide synthesis, as known in the art.

Immunisation against GST-VP1 and the GST protein alone will be made in BB rats by subcutaneous injections with 200 μg doses of the individual proteins in Freund's adjuvant followed by booster doses. BB rats selected for breeding will be immunised as adults at days 0, 10 and 30. Diabetes prone offspring to immunized parents will be followed until 70 days of age. At this time clinical signs of diabetes will be recorded before sacrificing the animal. Post mortem formalin fixed pancreas will be investigated for presence of LV VP1 antigen using IHC. In separate experiments diabetes prone offspring will be immunised as described above days 25, 32 and 45. These animals will also be followed until 70 days of age and tested for clinical signs of diabetes and presence of viral antigen in pancreas tissue.

Absence of clinical signs of diabetes at day 70 and/or absence of viral antigen detected by LV VP1 antisera will we regarded as successful immunization.

Immunisation for treatment or prevention of a picornavirales infection in a subject may also be performed using a pharmaceutically acceptable adjuvant, as described elsewhere herein.

### Example 7: Cross-Reactivity Between LV VP1 and DWV

Tissues from bees infected with Deformed Wing Virus and bees from a colony free of DWV where analyzed for presence of LV antigen using IHC previously described, with minor modifications (Shi, 1997). Presence of LV specific antigen was visualized using a polyclonal antiserum against recombinant LV immune dominant capsid viral protein 1 (VP1) raised in a rabbit (Tolf, 2008). As control we used serum from a rabbit immunized using the same protocol but with the carrier protein only.

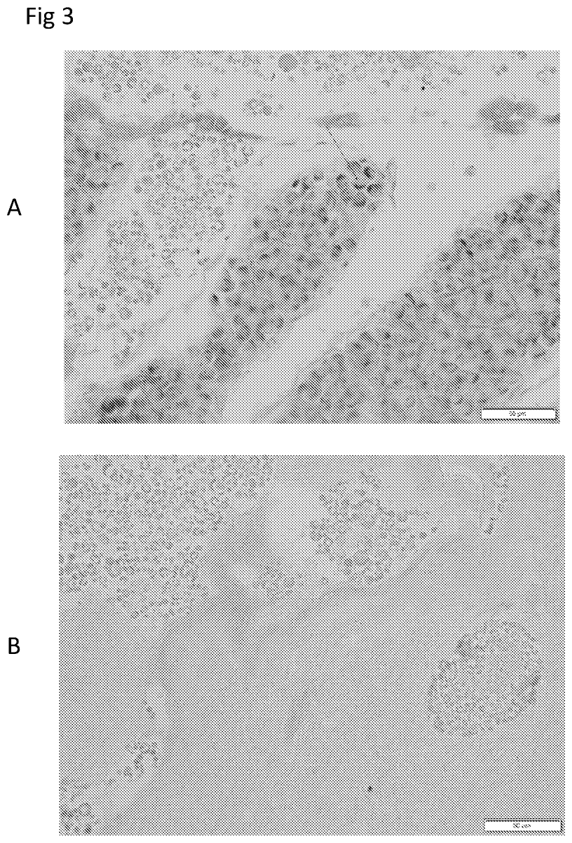

Tissues in bees infected with DWV stained positive, cf. FIG. 3A. Control antibody showed no reactivity (data not shown) and bees from a colony without DWV virus infection was IHC negative, cf. FIG. 3B.

This example indicates that viruses falling within the definition of Ljungan-like virus may be distantly related to Ljungan virus and may be considered as belonging generally to the order picornavirales, not restricted to picornaviridae and parechovirus.

### REFERENCES

Donoso Mantke et al. (2007). A new quantitative real-time reverse transcriptase PCR assay and melting curve analysis for detection and genotyping of Ljungan virus strains. Journal of Virological Methods, 141, 71-77.
Holmberg et al. (2009). Antiviral treatments reduce severity of diabetes in Ljungan virus-infected CD-1 mice and delay onset in diabetes-prone BB rats. Microbiol Immunol., 53(10), 567-572.
Lee et al. (2017). Antiviral Activity of Itraconazole against Echovirus 30 Infection In Vitro. Osong Public Health Res Perspect, 8(5), 318-324.
Niklasson et al. (2003). Development of type 1 diabetes in wild bank voles associated with islet autoantibodies and the novel Ljungan virus. Int J Exp Diabesity Res, 4, 35-44.
Niklasson et al. (2016). Diabetes Prevention Through Antiviral Treatment in Biobreeding Rats. Viral Immunology, 29(8), 1-7.
Pevear et al. (1999). Activity of Pleconaril against Enteroviruses. Antimicrobial Agents and Chemotherapy, 43(9), 2109-2115.
Shi et al. (1991). Antigen retrieval in formalin-fixed, paraffin-embedded tissues. J Histochem Cytochem, 39, 741-748.
Shi, S. (1997). Antigen retrieval immunohistochemistry: past, present, and future. J Histochem Cytochem, 45(3), 327-343.
Tolf, C. (2008). Characterization of polyclonal antibodies against the capsid proteins of Ljungan virus. J Virol Methods, 150(1-2), 34-40.
Ulferts et al. (2016). Screening of a Library of FDA-Approved Drugs Identifies Several Enterovirus Replication Inhibitors That Target Viral Protein 2C. Antimicrobial Agents and Chemotherapy, 60(5), 2627-2638.

1. A method for treatment or prevention of a picornavirales infection in a mammalian subject comprising administering a combination of a picornaviridae capsid binding anti-viral agent and at least one further anti-viral agent to said mammalian subject, with the proviso that when the picornaviridae capsid binding anti-viral agent is pleconaril then the further anti-viral agent is not solely ribavirin or solely (3,4-dimethoxyphenyl)-N-[(4-fluorophenyl)methyl]-2,5-dimethyl-pyrazolo[1,5-a]pyrimidin-7-amine.

2. The method according to claim 1, wherein the picornaviridae capsid-binding anti-viral agent is selected from the group consisting of pleconaril, vapendavir, and pocapavir.

3. The method according to claim 1, wherein at least one further anti-viral agent is selected from the group consisting of efavirenz, fluoxetine, amantadine, formoterol, carvedilol, itraconazole, and kinase inhibitors.

4. The method according to claim 1, wherein the combination comprises at least pleconaril, efavirenz and ribavirin.

5. The method according to claim 1, wherein the infection is caused by a virus causing in vivo-expression of a VP1-protein detectable in infected tissue through immunohistochemical staining using an antibody raised against the VP1-protein of Ljungan virus, while said virus is not detectable in said infected tissue using an RT-PCR assay adapted for detection of Ljungan virus.

6. The method according to claim 1, wherein the mammalian subject is of a species selected from the group consisting of human, cat, dog, pig, horse, cow, and camel.

7. The method according to claim 1, for use in a method for treatment or prevention of a picornaviridae infection, such as a parechovirus infection.

8. A method for treatment of a picornavirales infection in a mammalian subject comprising administering an anti-viral agent to said mammalian subject, wherein the picornavirales infection is caused by a virus causing in vivo-expression of a VP1-protein detectable in infected tissue through immunohistochemical staining using an antibody raised against the VP1-protein of Ljungan virus, while said virus is not detectable in said infected tissue using a PCR assay adapted for detection of Ljungan virus.

9. The method according to claim 8, wherein the anti-viral agent is selected from the group consisting of picornaviridae capsid binding anti-viral agents, including pleconaril, vapendavir, and pocapavir; efavirenz; fluoxetine, amantadine, formoterol, carvedilol, itraconazole, ribavirin, kinase inhibitors, and any combination thereof.

10. The method according to claim 8, wherein the mammalian subject is of a species selected from the group consisting of human, cat, dog, pig, horse, cow, and camel.

11. The method according to claim 1, wherein the picornavirales infection is an infection of the central nervous system, pancreas, liver, endocrine tissue, vascular tissue and/or muscle tissue of a subject.

12. The method according to claim 1 wherein the infection is of the central nervous system and the subject suffers from a neurological disease.

13. The method according to claim 12, wherein the neurological disease is Alzheimer disease, Multiple Sclerosis, Amyotrophic Lateral Sclerosis, Chronic Fatigue Syndrome, frontal lobe dementia, or Parkinson's disease.

14. The method according to claim 1. wherein the infection is of the pancreas and the subject suffers from Type 1 Diabetes or Type 2 Diabetes.

15. The method according to claim 1. wherein the infection is of the pancreas and the treatment or prevention of the picornavirales infection is for preventing or delaying onset of Type 1 Diabetes.

16. The method according to claim 1. wherein the infection is of the liver and the subject suffers from hepatitis.

17. The method according to claim 1. wherein the infection is of endocrine tissue and the subject suffers from dysregulation of endocrine function.

18. The method according to claim 1 wherein infection is of myocardium and the subject suffers from heart muscle disease.

19. The method according to claim 1, wherein the picornavirales infection is an infection of the central nervous system, pancreas, liver, endocrine tissue, vascular tissue and/or muscle tissue of a subject and the combination comprises pleconaril and at least one of efavirenz and carvedilol; and optionally ribavirin.

20-22. (canceled)

23. A pharmaceutical composition comprising Ljungan virus VP1 protein and a pharmaceutically acceptable adjuvant.

24. A method of treatment or prevention of an infection, in a mammalian subject, by a virus causing in vivo-expression of a protein detectable in infected tissue through immunohistochemical staining using an antibody raised against the Ljungan virus VP1 protein, while said virus is not detectable in said infected tissue using a PCR assay adapted for detection of Ljungan virus, said method comprising administering a Ljungan virus VP1 protein, or the pharmaceutical composition according to claim 23, to a subject in need thereof.

25. The method according to claim 24, wherein the mammalian subject is of a species selected from the group consisting of human, cat, dog, pig, horse, cow, and camel.
